(19) United States
(12) Patent Application Publication
(10) Pub. No.: US 2007/0075273 A1
(43) Pub. Date: Apr. 5, 2007

(54) Particle therapy procedure and device for focusing radiation

(76) Inventors: Denis Birgy, Grossenseebach (DE); Harald Breuninger, Eggolsheim (DE); Peter Frankenbach, Eltville (DE); Lothar Rossler, Reckenroth (DE)

Correspondence Address: BRINKS HOFER GILSON & LIONE, P.O. BOX 10395, CHICAGO, IL, 60610, US

(21) Appl. No.: 11/520,949

(22) Filed: Sep. 13, 2006

Related U.S. Application Data
(60) Provisional application No. 60/717,833, filed on Sep. 16, 2005.

(30) Foreign Application Priority Data
Sep. 16, 2005 (EP) EP 05020254

(51) Int. Cl. A61N 5/00

(52) U.S. Cl. 250/492.3

(57) ABSTRACT

A particle therapy system procedure and device for focusing radiation are provided. The particle therapy system comprising an acceleration unit that accelerates particles and a particle beam feed unit that directs particles to at least one radiation treatment location, an accelerator control unit that sets and controls the parameters of the acceleration and particle beam feed unit needed for irradiation, and an assignment unit that assigns and monitors a particle beam along a beam path in the acceleration unit and particle beam feed unit to a radiation treatment location requesting the particle beam.

The present patent document claims the benefit of the filing date under 35 U.S.C. § 119(e) of Provisional U.S. patent application Ser. No. 60/717,833 filed on Sep. 16, 2005, which is hereby incorporated by reference. This application also claims the benefit of EP05020254, filed Sep. 16, 2005.

### BACKGROUND

1Field

The present embodiments relate to a particle therapy system procedure and device for focusing radiation.

2Related Art

A particle therapy system generally includes a particle accelerator unit, a particle beam feed unit connected downstream and a number of radiation treatment stations. The acceleration of the particles, for example, protons, pions, helium, carbon or oxygen ions, is undertaken with the aid of a synchrotron or cyclotron. The accelerated high-energy particles are decoupled from the particle accelerator unit and directed into the particle beam feed unit (also referred to as the high energy beam transport system HEBT). When a synchrotron is used, the decoupling is undertaken, for example, via a KO exciter. The HEBT feeds the high-energy particles to the radiation treatment station at which a radiological process is to take place at that particular time.

For example, tumor therapy of a patient takes place at a radiation treatment station also referred to as a treatment station. The patient is positioned in the particle beam path and subjected to the high-energy particles. A “fixed beam” treatment chamber or station has particles arrive at the treatment station from a fixed direction. A “gantry-based” treatment chamber or station has a particle beam from different directions onto the treatment station of the gantry. The beam quality is monitored at a radiation treatment station referred to as the monitoring station. At the monitoring station, beam parameters, for example, particle energy, energy distribution and beam intensity are monitored using quality measurements.

High demands are made on the safety of a particle therapy system. The particle beam is fed to one radiation treatment station which is prepared for a radiological process and which has requested the particle beam. The particle beam should have the correctly requested parameters. A rapid interruption of the particle feed is needed in emergencies. The HEBT has a baffle, for example, that allows the particle beam to be rapidly deactivated. Generally, a control and safety system of the particle therapy system guarantees that the particle beam characterized with the necessary parameters is directed into the appropriate treatment area.

The parameters are defined in what is known as the treatment plan (therapy plan). The treatment plan specifies how many particles are to hit the patient, from which direction, and with what energy. The energy of the particles determines the penetration depth into the patient. For example, the location where the maximum dose is deposited is the location at which the maximum interaction with the tissue occurs during particle therapy. The parameters required by the treatment plan are generally converted by an accelerator control unit into setting parameters, for example, into the form of machine parameters, for the particle accelerator unit and the particle feed unit. The information as to the radiation treatment chamber to which the particle beam is to be directed is converted into setting parameters for the particle beam feed unit. A control unit of the radiation treatment station controls a positioning device, for example, with which a patient or a phantom is positioned in relation to the particle beam.

A particle therapy system with a number of fixed-beam treatment stations and a gantry is disclosed in EP 0 986 070. Different irradiation systems and techniques are described by H. Blattmann in “Beam delivery systems for charged particles”, Radiat, Environ, Biophys, (1992) 31:219-231.

A method for selecting a treatment area is known for example from U.S. Pat. No. 5,260,581 and a control and safety system for a beam therapy system is disclosed in U.S. Pat. No. 5,895,926.

### SUMMARY

The present embodiments are directed to a particle therapy system procedure and device for focusing radiation, which may obviate one or more of the problems due to the limitations and disadvantages of the related art.

A direct and permanently-assigned signal connection is, for example, a direct hardware connection. In one embodiment, the direct hardware connection is a single and safety-oriented signal line. A plurality of cable sections clamped together, with a single, continuously laid cable is used.

A direct and permanently-assigned signal connection or link provides a unique assignment of an element to a signal output. One element is always activated without a confirmation being needed here for verification of the activation of the correct elements via a, for example, protocol. The element may be set without any additional verification step. A process hardware-encoded and controlled in this manner allows setting of elements. For example, the elements are set for an HEBT that safely feeds a particle beam along a particle path defined by the elements to the requesting radiation treatment station. The signal connection is used in one direction in one embodiment, so that an error-prone logic system is not needed for differentiating the direction of the signal transmission.

Conventionally, a method, as described in U.S. Pat. No. 5,895,926, used a pure bus solution onto which variable signaling can be imposed. Hardware coding permits a safe allocation and/or a safe beam availability control and/or a safe setting of the beam path by dedicated hardware signal lines.

In one exemplary embodiment, an inventive particle therapy system includes an accelerator and particle beam feed unit that accelerates particles and directs particles from the accelerator to at least two radiation treatment stations. For example, this type of unit comprises a cyclotron or a synchrotron as an accelerator into which pre-accelerated particles are coupled. The particle beams are fed with the aid of at least one adjustable element in the beam path. The element or elements are set with the aid of the accelerator control unit corresponding to the beam path needed in each case. The setting parameters are transferred and stored in, for example, a buffer.

The particle therapy system includes an assignment unit that assigns and monitors correct particle beam guidance along the beam path. The particle beam travels within the accelerator and particle beam guidance unit to a radiation treatment station which is requesting the particle beam. The assignment unit is, for example, a safety-oriented and stored-program control unit.

To transfer an activation signal, a signal output of the assignment unit is connected via a direct and permanently-assigned signal connection to at least one of the settable elements. The setting parameter transmitted is only implemented in the element when an activation signal is present. For example, the activation signal is present before and/or during the implementation for example.

In one embodiment, the settable elements are deactivated as a matter of priority, for example, the assignment unit acts as a locking mechanism. For example, only if the activation signal is present are the appropriate currents set. Deactivation means, for example, that the default values are set. For example, there is no current flow in the magnet coils.

In one embodiment, the accelerator and particle beam feed unit include a plurality of elements. The plurality of elements are connected individually in each case via one permanently-assigned signal connection directly to one signal output of the assignment unit. The settable elements are connected via a direct hardware connection, for example, through individual, direct signal lines, to the assignment unit.

In one embodiment, the settable elements are, for example, beam deflection magnets that deflect the particle beam from the beam feeding system into the individual treatment rooms, a beam decoupling unit of an accelerator, for example a Knock Out exciter of a synchrotron ring, or a dipole magnet of a baffle in the HEBT. Possible setting parameters are, for example, the magnetic field, a current value to be set or an HF coupling-out frequency. A settable element is preferably embodied for processing and, depending on the presence of the activation signal for implementation of the at least one setting parameter transferred. In one embodiment, the settable element includes a buffer, for example, in which a transmitted setting parameter can be stored and read out after the activation signal is received.

In one embodiment, the setting parameters are transferred over a data bus system, to which the accelerator control unit and the relevant elements are linked. The setting parameter is defined by the irradiation process taking place at the radiation treatment station. In one embodiment, a control unit or one of the radiation treatment stations are linked to this data bus system or to a separate data bus system, for example, for exchange of the parameters of the particle beam and/or parameters of the acceleration and particle beam feed unit needed for irradiation. Examples of radiation treatment stations are a treatment station for radiotherapy, for example, a fixed-beam or gantry treatment station or a monitoring station for checking parameters on which the particle irradiation is based.

In one embodiment, a method for setting a beam path includes, for example, sending at least one setting parameter to the element. Sending an activation signal via a direct and permanently-assigned signal connection from the assignment unit to the element. This signal can be present for a short period or for the entire irradiation process. In one embodiment, the activation signal effects a switchover of a setting of the element, for example, to “able to be set”. In one embodiment, an activation signal must be present or must have been present during the setting to enable the setting of the setting parameter to be implemented. If the setting parameter and the activation signal are present, the setting parameter is converted in the element to the desired setting and mode of operation of the element. The acts may be executed in turn or possibly simultaneously.

Further possible processes for requesting a beam at a radiation treatment station can involve the following steps: sending a request signal from one of the radiation treatment stations to the assignment unit, for example, via a direct and permanently-assigned signal connection. Checking the availability of the particle beam, and, if the particle beam is available, allocating it to the requesting radiation treatment station. A confirmation signal is sent to the requesting radiation treatment station, for example, via a direct and permanently-assigned signal connection or via a data bus system. If an error occurs, the particle acceleration and/or a forwarding of the particle for example from the assignment unit and/or the control unit may be interrupted. For example, if an activation signal must be present continuously for setting the element, this activation signal is ended. If the activation signal acts as a switch, the element is switched to “not able to be set”.

### BRIEF DESCRIPTION OF THE DRAWINGS

FIG. 1 illustrates an exemplary therapy system,

FIG. 2 is a schematic of a safety-oriented switch unit according to one embodiment, and

FIG. 3 illustrates safety-oriented links according to one embodiment.

### DETAILED DESCRIPTION

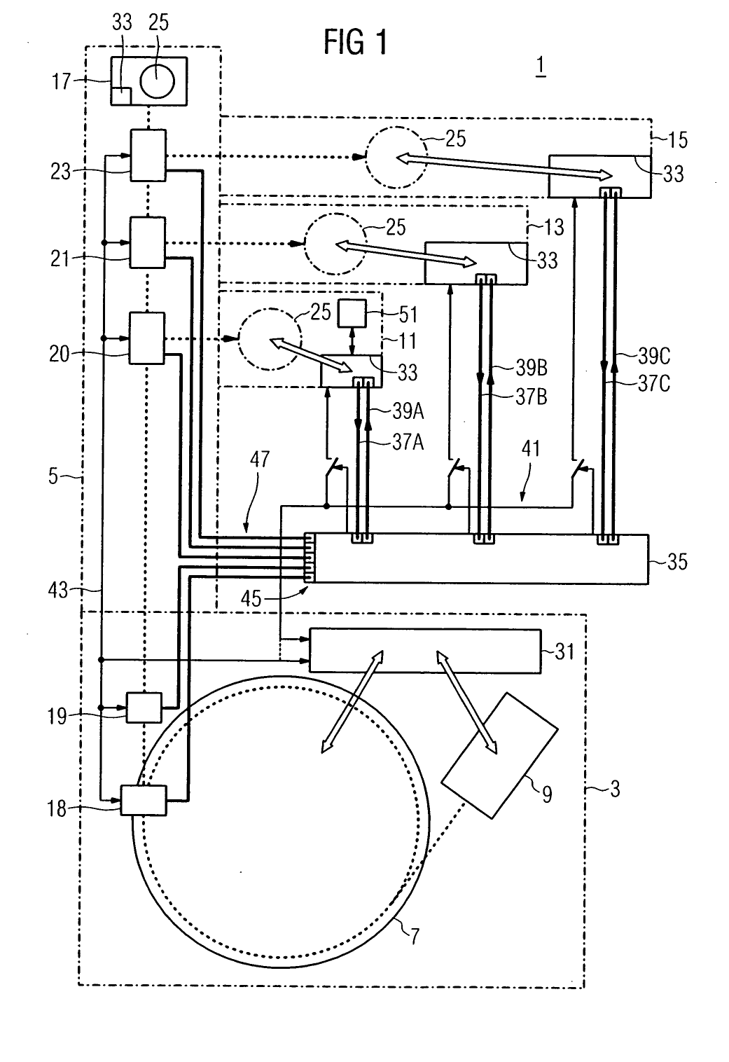

FIG. 1 shows a schematic diagram of a particle therapy system 1 and illustrates the interaction of the different control units. The control units effect and monitor the setting of components in order to send a beam with corresponding parameters to a radiation treatment station or location. Important signals are transmitted over a hardware connection not susceptible to errors. The hardware connection includes, for example, a single, separate specific line and is uniquely assigned to the transmission of a signal.

In one embodiment, the therapy system 1 includes an accelerator unit 3 and a particle beam feed unit 5. For example, a synchrotron 7 is used with an upstream linear accelerator unit 9. The particle beam feed unit 5 distributes the particle to a number of radiation treatment locations. As shown in FIG. 1, three treatment stations 11, 13 and 15 for radiotherapy and a checking station 17 monitor the quality of the particle beam. Quality assurance with the aid of quality procedures is undertaken at the checking location or station 17. The quality procedures use regular tests that verify the previously defined beam parameters, for example, position and intensity levels, of particle energies. Intensity levels are contained in a library and are checked using phantoms by automated Bragg peak measurements.

A decoupling device 18 decouples and directs the particles stored, for example, in the synchrotron ring 7 into the particle beam feed unit 5. A baffle 19 includes three small dipole magnets arranged after the extraction unit 18. The baffle 19 provides rapid beam deactivation after ending or interruption of the irradiation process. For example, when there is rapid deactivation of the center dipole, the beam is destroyed at one collimator.

In one embodiment, the particles are fed to the radiation treatment stations 11, 13 and 15 by deflecting the particle beams using deflection magnets 20, 21 and 23 from a main beam direction in the beam feed unit 5. The checking station 17 is located in the main direction of the beam. At the radiation treatment stations, the particles interact with a patient to be irradiated in irradiation zones 25. One of the irradiation zones 25 is, for example, a maximum scannable scan area of a (raster) scan device, a maximum irradiatable scatter area of a scatter device or a gantry irradiation area that can be set.

In one embodiment, the linear accelerator unit 9 includes one or more drivable ion sources, a low-energy beam guidance system, a radio-frequency quadrupole, a drift tubular accelerator and an injection beam guidance system. The linear accelerator unit 9, for example, creates one or more particle types, frees the particle types from contaminations of undesired types of particle, and sets the beam intensity in the low-energy range. For example, for the synchrotron, the linear accelerator unit 9 pre-accelerates the particles and sets the pulse length and the beam parameters in accordance with the requirements of the synchrotron.

In one embodiment, a scanning method is used for irradiation in the therapy system 1. A slow extraction facilitates an optimum use of the accelerated particles and precise beam monitoring during the tumor scanning. For example, a HF knock out method is used. The knock out exciter forms the decoupling unit 18.

In one embodiment, the control and safety system of the therapy system 1 according to FIG. 1 is divided into a number of components. Alternatively, the system is divided up in another way or not at all, provided the different aspects are taken into account in the monitoring.

As shown in FIG. 1, an accelerator control unit 31 insures that the requested particle beam arrives in the treatment room in accordance with its specification. Control units 33 arranged at the radiation treatment stations control the execution sequence of an irradiation process and ensure that the particle beam hits a patient according to the treatment plan.

In one embodiment, the control and safety system includes an assignment unit 35. The assignment unit 35 assigns a particle beam to the irradiation location 11, 13, 15 for which a beam is requested. This assignment insures that a particle beam is directed only to the irradiation location 11, 13, 15 for which a beam is requested. To this end the assignment unit 35 is connected to control units 33 at least to transfer of a request signal over a permanent or fixed and uniquely-assigned signal line 37A, 37B, 37C. In one embodiment, a further permanently-assigned signal line 39A, 39B, 39C exists between the assignment unit 35 and the control units 33. For example, a confirmation signal is transferred on the further permanently-assigned signal line 39A, 39B, 39C from the assignment unit 35 to that radiation treatment station to which the particle beam is next supplied.

In one embodiment, the control and safety system includes at least one data bus system 41 to which the control units 33 and the accelerator control unit 31 are linked. For example, the at least one data bus system 41 transfers setting parameters to the accelerator unit 3 and the particle beam feed unit 5 for a next irradiation to be performed. The assignment unit 35 acts on the data bus system 41, so that only that radiation treatment station 11, 13, 15 which has received a confirmation signal can transfer parameters.

The accelerator control unit 31, elements of the acceleration unit and particle beam feed unit that are settable are linked to a further data bus system 43 connected to the data bus 41 (dashed connection). For example, in the embodiment shown in FIG. 1, the decoupling unit 18, the chicane 19 and the deflection magnets 20, 21, 23 are linked to the further data bus system 43. The setting parameters are transmitted to the elements via the data bus system 43. The elements set the currently requested particle beam path and transport the particles with the, for example, correct energy. The specific setting parameters of the elements in the high-energy beam path are determined as a function of a specified irradiation location.

In one exemplary embodiment, the setting parameters are implemented if an activation signal of the assignment unit 35 is present at the element to be set. The settable elements are connected to signal outputs 45 of the signal assignment unit 35 via direct, permanently-assigned signal lines 47.

In one embodiment, request and/or activation signals are sent and received via specific unambiguous hardware connections. It is not possible to send signals from other irradiation locations or conveyed to other elements erroneously. The request signal is sent from certain and known radiation treatment station and/or only certain activating elements are set for defining the beam path.

An irradiation of a patient is performed, for example, using a system as shown in accordance with FIG. 1. The irradiation includes required parameters, for example, beam incidence direction, beam intensity, particle type, and particle energy. These parameters are defined in a treatment plan 51.

For example, the treatment plan for the patient is loaded at the radiation treatment station, all technical safety requirements fulfilled and the patient is positioned accordingly. A therapy control system 1, for example, a control unit 33 of the radiation treatment stations 11, 13, 15, requests a beam with the planned parameters for the current radiation treatment station. Only tested and released data records of parameters present as stored data in the accelerator control system 31 are used and requested.

An operator initiates the transmission of a request signal from, for example, the control unit 33 of the radiation treatment station 11 along the direct permanently-assigned signal lines 37A to the assignment unit 35. The assignment unit 35 checks the availability of the particle beam. The assignment unit 35 only assigns the particle beam to the requesting treatment room once this irradiation process has ended. If an irradiation process is being performed at an adjacent radiation treatment station then the assignment unit 35 does not assign the particle beam. For example, the assignment unit 35 only releases the connection from the control unit 33 of the treatment room 11 to the accelerator control unit 31 in the data bus system 41 for the transfer of the desired parameters for the subsequent irradiation process.

The assignment unit 35 sends activation signals to the settable elements via the permanently-assigned signal lines 47. In one embodiment, the settable elements are, for example, the decoupling unit 18, the baffle 19 and the deflection magnet 20. The accelerator control unit 31 sets parameters in these elements. The setting parameters transferred by the accelerator control unit 31 are implemented in the elements and define the particle beam path required only if the activation signal is available. The settable elements are deactivated as a matter of priority. Only if an activation signal is present are the, for example, appropriate currents set. Deactivation means that for example the default value “current to zero” is set.

The assignment unit 35 transmits a confirmation signal along the connecting line 39A. Particles are supplied for irradiation in the irradiation area 25 after confirmation of the confirmation signal by the radiation treatment station 11.

Except for the presence of an activation signal for the actual implementation of physical settings, the order of setting processes and signal transfers is interchangeable. In an alternate embodiment, directly after the assignment of the particle beam to the treatment room 11, the confirmation signal is transmitted to the treatment room 11 along the connecting line 39A. An active “beam on” activation signal from the assignment unit 35 is triggered by the control unit 33 of the treatment room in response to the confirmation signal as well as the transfer of setting parameters from the accelerator control unit 31 to the relevant elements. Subsequently, the setting parameters in the elements are physically converted and the particles are supplied to the radiation treatment station. In this embodiment of the execution sequence, the conversion is only performed after a confirmation signal is received. A possible incorrect setting is avoided at an early stage. For example, when a non-requesting control unit 33 receives a confirmation signal, deactivation is performed automatically.

In this embodiment, the process is subdivided into three stages. In a preparation stage, only the control unit and the assignment unit communicate (beam request signal, confirmation signal of beam assignment and also “beam on” signal. In a setting stage, the assigned control unit and the accelerator control unit communicate, for example, corresponding beam parameters are requested and the corresponding parameters are transferred to the elements and the accelerator unit. In an activation stage, the assignment unit communicates directly with the elements and makes the elements adjustable, so that the parameters transferred by the accelerator control unit can be converted. The activation stage makes the setting physically possible and implements the setting. In one embodiment, the activation stage takes place at the same time as the second stage.

The therapy system operates entirely autonomously during the irradiation process. For example, the control unit 33 controls scanner magnets and beam diagnosis units that monitor the beam quality. The only intervention option for the operating personnel is to abort the irradiation process. The assignment unit 35 withdraws the permission to be activated via the direct and permanently-assigned signal lines to the elements when a beam abortion is initiated or an error type is detected in another way in the system. In one embodiment, the beam is destroyed, for example, within the baffle 19 by shutting off a dipole magnetic field. The deflection magnets 20, 21, 23 are switched to zero-current for example, and the KO exciter is switched off.

In one embodiment, after an irradiation process is completed, the settable elements are set back to their default values. The deflection and/or baffle magnetic fields are powered down to zero, and the frequency is switched off. In one embodiment, during interactions with an operating system of the therapy system optimizing usage for control of irradiation processes arising, a default value is skipped because of the next irradiation process taking place through corresponding control of the assignment unit. The beam path is rapidly available for the following irradiation process.

The tasks of the different components of the control and safety system for the beam request and beam path definition can be summarized in some embodiments as follows: The accelerator control unit 31 controls the correct values of the setting parameters for the adjustable elements in the acceleration unit and particle beam feed unit. The assignment unit 35 insures that these parameters are set by means of an activation process, in which only those elements are explicitly activated that are necessary for a beam path. The assignment unit has stored a table with the possible beam paths in a, for example, look-up table. In addition the availability of the particle beam is checked within the assignment unit 35. The assignment unit 35 includes a safety-oriented programmable logic controller. The particle beam is only allocated if it is available. The control units in the radiation treatment stations supply the data from the radiation treatment station and in the final analysis decide on the supply of the beam, for example, they initiate the feeding of the beam to the corresponding radiation treatment station.

Use of the direct and permanently assigned links is not restricted to the embodiment outlined in FIG. 1. The link between one or more of the control units 33 and the assignment unit 35 can be embodied in an alternative way. For example, the control units 33 and the assignment unit 35 can be connected via an antenna receiver/transmitter.

The illustrated distribution is exemplary only. For example, different elements can be combined within one unit. In one exemplary embodiment, parameters for the therapy plan can be directed to the accelerator control unit 31 directly and not via a control unit 33 of a treatment chamber.

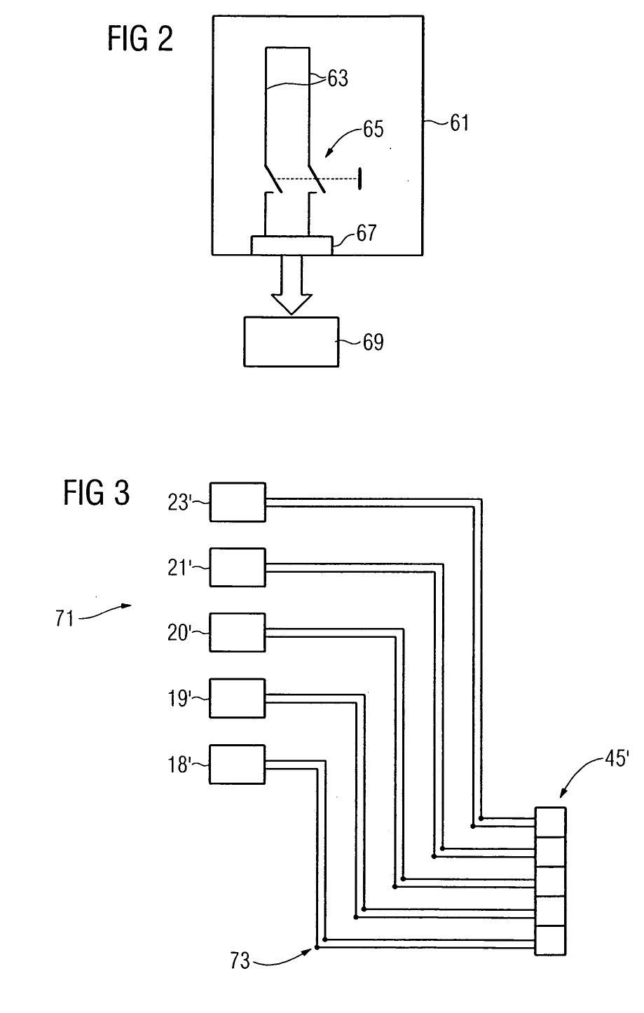

FIG. 2 illustrates an exemplary switch unit 61. The switch unit 61 can be used in, for example, the control unit 33 and/or the assignment unit 35. The switch unit 61 is used to convey a request signal, confirmation signal, and/or activation signal. In one exemplary embodiment, two leads 63 are connected in parallel. The two leads 63 are connected via a positive-opening/positive-closing switch 65 to a signal output 67. The switch 65 opens/closes the two leads 63 together and, in the event of a fault, assumes a safe state. The signal output is connected to a unit 69. For example, the unit 69 is one of the control units 33, the assignment unit 35, the decoupling unit 18, the baffle 19, or one of the bending magnets 20, 21, 23.

FIG. 3 illustrates the use of double leads for conveying activation signals to elements 71, for example, a decoupling unit 18′, a baffle 19′, and bending magnets 20′, 21′, 23′. The leads are applied by a corresponding switch unit to signal outputs 45′ which, according to FIG. 1, are part of the assignment unit 35. The use of clamping means 73 may in view of the size of a therapy system be unavoidable.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

1. A particle therapy system comprising:
an acceleration unit;
a particle beam feed unit operable with the acceleration unit, the particle beam feed unit operable to direct particles to at least two radiation treatment locations;
an accelerator control unit that is operable to set and control parameters of the acceleration unit and particle beam feed unit, and
an assignment unit that assigns and monitors a particle beam along a beam path in the acceleration unit and particle beam feed unit to a radiation treatment location requesting the particle beam,
wherein the acceleration unit and particle beam feed unit comprise at least one settable element that adjusts the beam path, and is connected to the accelerator control unit for transfer of at least one settable parameter,
wherein the at least one settable element is connected to a signal output of the assignment unit via a permanently assigned signal connection that receives at least one activation signal, and
wherein the at least one activation signal effects an implementation of the setting parameter in the element.

2. The particle therapy system as claimed in claim 1, wherein the signal connection is a direct hardware connection.

3. The particle therapy system as claimed in claim 1, wherein the acceleration unit and particle beam feed unit comprise a plurality of elements connected via a permanently assigned signal connection, and hardware-encoded to at least one signal output of the assignment unit.

4. The particle therapy system as claimed in claim 1, wherein the at least one settable element is a beam deflection magnet, a beam decoupling device of an accelerator unit, a KO exciter of a synchrotron ring, a deflection magnet downstream from the beam decoupling device, or any combination thereof.

5. The particle therapy system as claimed in claim 1, wherein the at least one settable parameter is a value of a magnetic field, a current value, decoupling frequency, or any combination thereof.

6. The particle therapy system as claimed in claim 1, wherein the at least one settable element is operable to process and implement the at least one settable parameter when at least one activation signal is present.

7. The particle therapy system as claimed in claim 6, wherein the at least one settable element comprises a memory operable to store the at least one setting parameter and operable to read out the at least one settable parameter for implementation after the activation signal is received.

8. The particle therapy system as claimed in claim 1, wherein the accelerator control unit and the at least one settable element are linked to each other, the link operable to transfer the at least one setting parameter to a first data bus system.

9. The particle therapy system as claimed in claim 1, wherein an accelerator control unit and at least one control unit of one of the radiation treatment stations are linked to the first or to a second data bus system, the first or second data bus system operable to exchange parameters of the particle beam, the acceleration unit and particle beam feed unit required for irradiation that are defined on the basis of a treatment plan, or both.

10. The particle therapy system as claimed in claim 1, wherein at least one of the radiation treatment locations comprises a treatment location at which a patient is irradiated with the particles.

11. The particle therapy system as claimed in claim 1, wherein at least one of the radiation treatment locations comprises a checking location operable to check a parameter characterizing a particle irradiation.

12. The particle therapy as claimed in claim 1, wherein the assignment unit comprises a programmable logic controller.

13. A method for setting a beam path to one of at least two radiation treatment stations of a particle therapy system, wherein the beam path is set in an acceleration unit and a particle beam feed unit using at least one settable element and an assignment unit, the method comprising:
sending at least one setting parameter to the at least one settable element,
sending at least one activation signal via a permanently assigned signal connection of the assignment unit to the at least one settable element, and
implementing the at least one setting parameter in the at least one settable element after the sending of the activation signal.

14. The method as claimed in claim 13, further comprising:
sending a request signal of one of the radiation treatment stations to the assignment unit via the permanently assigned signal connection before setting the beam path that defines the beam path to be set, and
checking an availability of the particle beam and conveying the result to the requesting radiation treatment station if the particle beam is available.

15. The method as claimed in claim 14, further comprising:
transferring parameters of the requested particle beam and of the beam path from a control unit of the radiation treatment station requesting the particle beam to an accelerator control unit that sets and controls the parameters needed for irradiation of the accelerator and particle beam feed unit after the allocation, and
sending the at least one setting parameter from the accelerator control unit to the at least one element.

16. The method as claimed in claim 15, further comprising: sending a confirmation signal to the requesting radiation treatment station via another permanently assigned signal connection or via a data bus system, to which the control unit and the assignment unit are linked after the allocation.

17. The method as claimed in claim 16, further comprising: sending the activation signal after the confirmation signal has been confirmed.

18. The method as claimed claim 13, wherein aborting the particle acceleration, forwarding of the particles, or both from the assignment unit, control unit, or both after a confirmation signal has been sent to a radiation treatment station that has not sent a request signal.

19. A method comprising using a permanently assigned signal connection between a settable element that sets a beam path in a particle therapy system and an assignment unit of a particle therapy system that transfers an activation signal.

20. A device for setting a beam path to one of at least two radiation treatment locations of a particle therapy system, wherein the beam path is set in an accelerator and particle beam feed unit of the particle therapy system by at least one adjustable element and an assignment unit that assigns and monitors a correct particle beam guidance, the device comprising:
a means for sending at least one setting parameter to the at least one element,
a means for sending an activation signal via a direct and permanently assigned signal connection of the assignment unit to the at least one element, and
a means for implementing the at least one setting parameter in the at least one element after the activation signal has been sent.

21. The device as claimed in claim 20, further comprising: a means for receiving a request signal, means for checking an availability of the particle beam and means for allocating the particle beam to the requesting radiation treatment station.

22. The device as claimed in claim 20, further comprising: a means for transferring parameters of the requested particle beam and of the beam path, from a control unit of the radiation treatment station requesting the particle beam to an accelerator control unit that sets and controls parameters of the accelerator and particle beam feed unit needed for irradiation.

23. The device as claimed in claim 20, further comprising: a means for sending a confirmation signal to the radiation treatment station, wherein the confirmation signal is sent via a second direct and permanently assigned signal connection or via a data bus system, to which the control unit and the assignment unit are linked.

24. The device as claimed in claim 20, further comprising: a means for interrupting particle acceleration, forwarding of the particles or both, controlled by the assignment unit, the control unit, or both.

25. The particle therapy system as claimed in claim 1, wherein the signal connection is an individual signal line.

26. The particle therapy system as claimed in claim 3, wherein the at least one element is a beam deflection magnet, a beam decoupling device of an accelerator unit, a KO exciter of a synchrotron ring, a deflection magnet downstream from the beam decoupling device, or any combination thereof.

27. The particle therapy system as claimed in claim 7, wherein the setting parameter is read from the memory for implementation after the activation signal is received.
